(19) United States
(12) Patent Application Publication
(10) Pub. No.: US 2018/0344974 A1
(43) Pub. Date: Dec. 6, 2018

(54) Catheterization with reduced catheter withdrawal damage

(71) Applicant: John T. R. Wilson, Seattle, WA (US)

(72) Inventor: John T. R. Wilson, Seattle, WA (US)

(21) Appl. No.: 15/998,284

(22) Filed: Jul. 30, 2018

Related U.S. Application Data
(63) Continuation-in-part of application No. 13/998,397, filed on Jan. 13, 2014, now abandoned.

(51) Int. Cl. A61M 25/00

(52) U.S. Cl. CPC A61M 25/0043; A61M 25/007; A61M 2025/0062; A61M 2025/0059; A61M 25/0074

(57) ABSTRACT

This invention concerns urinary catheterizing and identifies a cavitation occurring at catheter removal as the source of a damaging problem, and shows that a vent to atmosphere of the catheter tip prior to the catheter and vent removal prevents this problem. This invention disclosure shows the vent and method to prevent this problem.

The prior art for self-catheterizing specifies six catheterizes and catheter removals per day. Each catheter removal causes a small amount of damage, damage is cumulative and enlarges at subsequent catheterizes. After many catheterizes, the enlarged damage at catheter removal causes a serious bleeding problem.

This invention has two cooperative parts related in time as early and later. The early part inserts a lubricated catheter to urethra and bladder to remove urine, and the later part inserts a vent to a catheter urine discharge lumen and catheter tip to prevent this problem at the catheter and vent removal.

### CROSS REFERENCE

This application is a continuation-in-part of prior U.S. Non-Provisional patent application Ser. No. 14/756,602, filed Sep. 23, 2015 entitled “Reduced Cavitation in Catheter Removal”.

### BACKGROUND OF THE INVENTION

This invention relates to the human urinary track and to draining urine from the bladder by catheterizing, and the invention identifies a cavitation occurring at catheter removal from the urinary track as the source of a damaging problem. This invention discloses a method and apparatus to prevent this damaging problem.

The catheterizing includes two withdrawals: 1) withdrawal of urine from the bladder by a catheter, and 2) withdrawal of the catheter from the urinary track after urine drainage is completed. Also, the urinary catheterizing can be divided into two parts: 1) an early part and: 2) a later part. The early part consists of insertion of a catheter into the bladder to drain collected urine into a collector, such as a Pyrex pitcher. The two main points of the early part are: 1) the initiation of urine drain flow from the bladder to catheter tip through the catheter to the catheter urine exit to the collector, and: 2) the completion and stoppage of the bladder urine drainage. The two main points of the later part relate to withdrawal and are: 1) the potential for a cavitation transient at the initiation of catheter withdrawal, and: 2) the completion of catheter withdrawal and the viewing of the catheter tip after removal from the urethra for any evidence of bleeding.

Cavitation is a hydraulic term, often used in pump design and action, meaning the formation of a partial vacuum in a liquid and the subsequent collapse of the vacuum causing cavitation damage to solid surfaces. This invention identifies cavitation as a transient damaging problem in urinary catheterizing at the moment of catheter removal after catheterizing drainage is completed. This invention shows venting to atmosphere of the catheter tip prior to catheter removal prevents a transient partial vacuum formation of cavitation and thus prevents transient cavitation and the transient damage of this cavitation on solid surfaces enclosing or interfacing; then, the next catheter insertion disrupts any healing from the previous catheter-removal cavitation damage. Thus, the prior catheterizing art leads, after many catheterizes, to serious urinary bleeding. The self-catheterizing art specifies six to seven catheterizes per day.

In human catheterizing, bleeding is undesirable from any source in the urinary track. Sharp edges on the catheter can cause bleeding. Catheterizing without allowing sufficient time for healing of previous bleeding sources is undesirable.

Biopsies of bladder cells and at other urinary tract locations are done to identify bleeding sources. Biopsies have identified impact-damaged bladder cells as suspected bleeding sources.

### BRIEF SUMMARY OF THE INVENTION

The invention recognizes that damaging urinary cavitation may occur after human bladder catheterizing upon removal of a configured catheter, considered 17 inches long, and describes a method and devices to reduce or avoid that cavitation. This invention describes an apparatus and a method to reduce or avoid cavitation occurring at the tip of a 17 inch catheter containing residual urine and progressing into a configured urinary parts interface by venting air or other fluid to the tip of the configured catheter prior to or coincident with or during removal of the configured catheter following catheterizing. By admitting the atmospheric or outside air or other fluid to the tip of the configured catheter, no partial vacuum will be formed when the catheter is removed from the configuring urethra. The vent is 22 inches long.

### BRIEF DESCRIPTION OF THE DRAWINGS

This invention is described using eight Figures and include some enlargements for ease of presentation. FIG. 1 through FIG. 6 depict cross-sections through a configured urinary parts interface in various stages of catheterizing. The configured urinary parts interface is considered to include: urethra 2, sphincter 5, valve 6, bladder 4, left-kidney ureter 7 and right-kidney ureter 8. FIG. 7 depicts a catheter. FIG. 8 depicts a vent line or vent. The FIG. 1 and FIG. 2 depict the initial stages of catheterizing; these FIGS. 1 and 2 are considered Prior Art. The FIG. 3 and FIG. 4 depict stages of catheterizing following the FIG. 2 stage without using this invention; these FIGS. 3 and 4 are considered Prior Art. The FIG. 5 and FIG. 6 depict stages of catheterizing following the FIG. 2 stage using this invention.

FIG. 1 depicts bladder 4 being drained of urine 10 through catheter 1 with the valve 6 open. The catheter 1 is shown after being inserted into the urethra 2, using lubricant 3 and pushing valve 6 to open to the bladder 4. This FIG. 1 shows prior art.

FIG. 2 depicts sphincter 5 operated to close valve 6 to interrupt the flow path from the bladder 4 to the catheter 1 thus stopping the flow of urine 10. This FIG. 2 shows prior art. FIG. 2 also depicts some of configured urinary parts interface 9 between valve 6 and catheter 1.

FIG. 3 depicts the stage following the FIG. 2 stage without using this invention. FIG. 3 depicts urine 10 and the formation of a partial vacuum 11 in the configured urinary parts interface 9 and elsewhere at the start of catheter 1 removal. The partial vacuum 11 extends from the configured urinary parts interface 9, through the valve 6 and into the bladder 4.

FIG. 4 depicts the stage that follows the FIG. 3 stage without using this invention. FIG. 4 depicts the collapse of the partial vacuum 11 into pieces trapping urine 10 drops within the vacuum pieces 11 and impacting these pieces, onto solid surfaces of bladder 4, valve 6 and ureters 7 and 8. FIG. 4 is prior art.

FIG. 5 depicts the stage following the FIG. 2 stage using this invention. FIG. 5 also depicts this invention with the introduction of vent 12 into the urine flow path of catheter 1 to the tip of catheter 1. The vent 12 position at the tip of catheter 1 may be determined by the blockage from the catheter 1 tip from farther vent 12 movement along the flow path toward the bladder or by other means. One other means to determine the position of vent 12 is to use a coude catheter and use the coude catheter elbow to block farther vent 12 movement toward the bladder. The vent 12 is a flexible plastic line 22 inches long of 0.065inches diameter. FIG. 5 also depicts the sphincter 5 operated to close valve 6 at catheterizing completion to interrupt the flow path from the bladder 4 to the catheter 1 thus stopping the flow of urine. FIG. 5 also depicts the introduction of atmospheric air 13 through and around the vent 12 at catheterizing completion.

FIG. 6 depicts the stage following the FIG. 5 stage using this invention. FIG. 6 also depicts the catheter 1 partially removed from the configured urinary parts interface 9 with no partial vacuum formation, nor vacuum collapsing, nor urine 10 in vacuum pieces, impacting solid surfaces of bladder 4, valve 6, urethra 2, or ureters 7 and 8.

From the stage depicted in FIG. 6 to complete one catheterizing, the configured catheter 1 and vent 12 would be simultaneously fully withdrawn from the configuring urethra 2 having suppressed any transient partial vacuum formation, transient cavitation, or cavitation damage.

FIG. 7 depicts a catheter 1 in approximately full size. The tip of the catheter 1 is at the left end and has the rounded point. This tip of a coude catheter has an elbow with a sharp elbow curvature about one inch from the rounded tip end and is called a coude catheter. The tip is inserted in the urethra 2 in catheterizing using this invention, as depicted in FIG. 5, and in FIG. 7 the tip has the elongated opening outline, which is the entry for urine from the bladder 4. During catheterizing, the outlet of urine from the catheter is from the squared end, also called the discharge end or the catheter urine exit. The length of the catheter 1 is considered to be 16 to 17 inches.

FIG. 8 depicts a vent 12 in approximately full size. The length of the vent 12 is considered to be 22 inches.

### DETAILED DESCRIPTION OF THE INVENTION

The invention will be described by describing the drawings in more detail in a catheterizing sequence.

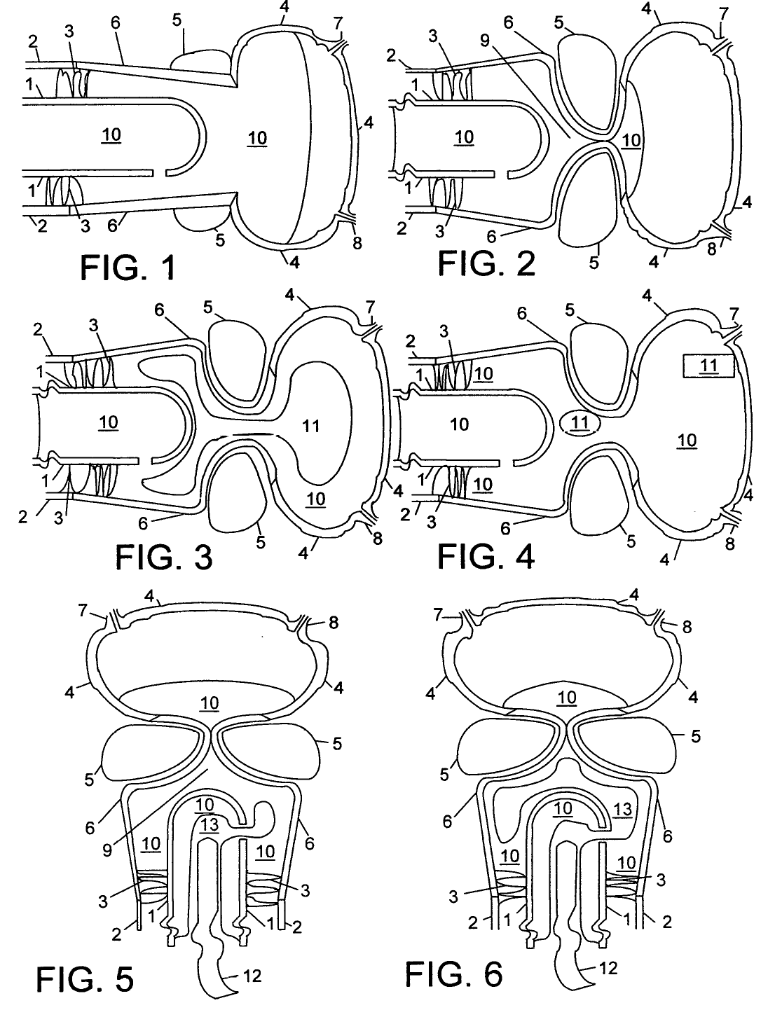

In FIG. 1 is depicted catheterizing in the process of draining urine 10 from the bladder 4 into the catheter 1 via the hole in catheter 1 in the inlet of catheter 1. Catheterizing starts when the catheter 1 and the mouth of urethra 2 have been coated with lubricant 3 and the catheter 1 has been inserted in the urethra 2 and pushed farther into the urethra to open the valve 6 and start the flow of urine 10. The catheter used is made of latex which is more rigid when cool and thus easier to insert and start the flow of urine when cool. Body heat will warm the catheter and make the catheter less rigid; so, the time from urethra insertion to the flow of urine should be reasonably quick. The lubricant 3 used is silica gel, and the commercial product is called “Surgilube”. FIG. 1 is prior art.

In FIG. 2 is depicted catheterizing in which the draining of urine 10 from bladder 4 has been determined to be completed and the sphincter 5 has constricted and closed valve 6. A small amount of urine 10 remains in bladder 4 and some urine 10 remains in the tip of catheter 1 and for some distance to the left in catheter 1. FIG. 2 is prior art.

The FIG. 3 depicts a catheterizing stage of catheter removal without the use of subject invention following a FIG. 2 stage. The FIG. 3 shows catheter 1 slightly withdrawn to the left causing the transient formation of a partial vacuum 11 in the configured urinary parts interface 9, previously shown in FIG. 2, and the transient partial vacuum 11 permeates the urine 10. FIG. 3 is prior art.

The FIG. 4 depicts a catheterizing stage following a FIG. 3 stage which includes the transient expansion of the transient partial vacuum 11 through valve 6 and into bladder 4. The transient partial vacuum 11 is depicted collapsed into pieces, rectangular and spherical, next to valve 6, bladder 4 and ureters 7 and 8. The urine 10 permeates the transient partial vacuum 11 pieces and as those transient vacuum pieces 11 collapse with urine 10 drops inside, the urine 10 drops are accelerated and make damaging impacts on enclosing or interfacing solid surfaces. This is called cavitation. This is what this invention prevents. FIG. 4 is prior art.

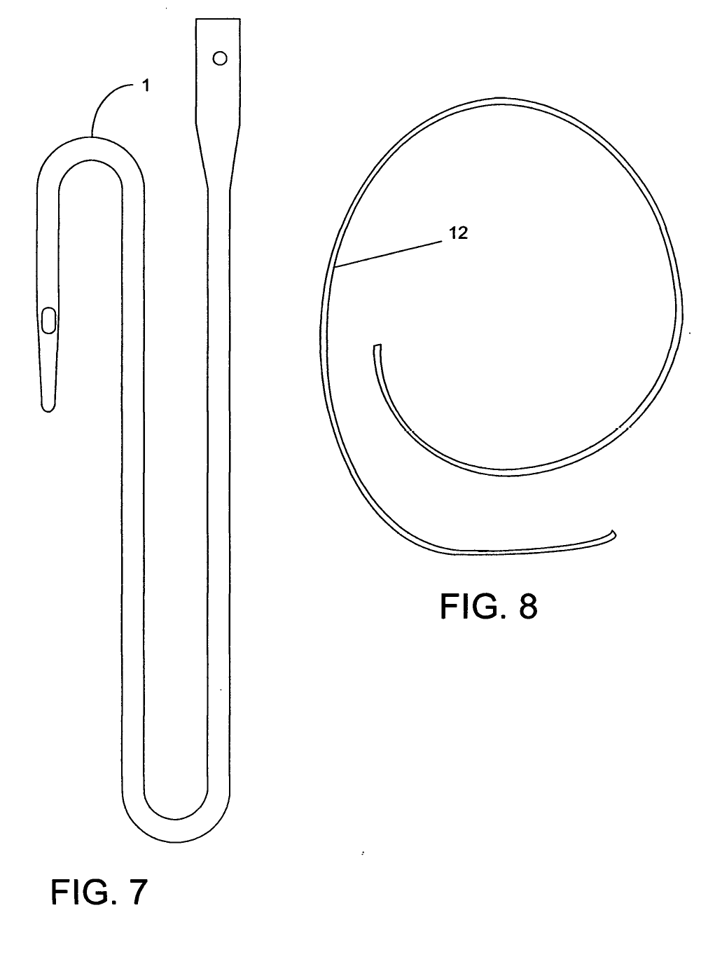

In FIG. 5 is depicted the stage following from the FIG. 2 stage using this invention, in which the vent 12 of this invention is inserted into the urine outlet mouth of catheter 1 after urine outflow has stopped. The vent 12 is a flexible plastic line which may be a tube. This vent 12 is inserted into the discharge end of catheter 1, shown as the squared end in FIG. 7, and pushed to the tip of catheter 1. The stiffness of vent 12 is enough to allow the vent 12 to be pushed in the catheter 1. By this action the atmospheric air 13 is introduced to the tip of catheter 1 and into the configured urinary parts interface 9. The presence of atmospheric air at the tip of catheter 1 prevents the formation of a partial vacuum at this place and in the surrounding places. The vent 12 used is a flexible plastic line longer than the catheter 1 for ease of manual manipulation and of a diameter to fit easily and move freely within the catheter 1 urine flow path. The ends of the vent 12 are filed to prevent gouging the inside diameter of the catheter 1.

The FIG. 6 depicts the stage following from the FIG. 5 stage using this invention. The FIG. 6 shows the catheter 1 and vent 12 slightly withdrawn from the configured urinary parts interface 9 causing a vacancy in the urine 10 in the configured urinary parts interface 9 which vacancy is filled by atmospheric air 13; as this configured catheter and vent 12 is withdrawn from configured urethra 2 there is no partial vacuum 11 formed and there is no cavitation nor is there any cavitation caused damage.

From the stage depicted in FIG. 6, to complete one catheterizing, the configured catheter 1 and vent 12, together and simultaneously, are fully withdrawn from the configuring urethra 2, separated, water rinsed and dried.

A working model of this invention has been made and tested. The catheter 1 has been a Bard 16 Fr. Coude tip, 16 inches long. The vent 12 has been an Arnold Trimmer line, 0.065 inches diameter and 22 inches long; the ends have been filed to prevent gouging the inside diameter of the catheter. The lubricant 3 has been silica gel, commercially Surgilube by HR Pharmaceuticals, Inc.

While certain preferred embodiments of the present invention have been disclosed in detail, it is understood that various modifications in its structure may be adopted without departing from the spirit of the invention or the scope of the following claims.

I claim as my invention:

1. An apparatus for urinary catheterizing without cavitation damage comprising a coude catheter externally lubricated with silica gel and a vent to the coude catheter tip elbow in a urinary parts region after urine drain completion prior to withdrawing the catheter and vent from the urethra thus completing a urinary catheterizing without cavitation damage.

2. A reduced cavitation method for a configured human urinary intermittent catheter with a coude catheter in thirteen steps as follows:
the first step is open an alcohol prep pad container;
the second step is to remove the gauze from the alcohol prep pad container and discard the gauze;
the third step is to water rinse the alcohol prep pad container to remove any residual alcohol;
the fourth step is to apply a silica gel lubricant dab of about 2 cm. diameter to the interior of the alcohol prep pad container;
the fifth step is to manually transfer a first portion of the silica gel lubricant dab from the alcohol prep pad container to the entire configured catheter external surface to within 2 inches of the squared end for urine exit;
the sixth step is to manually transfer a second portion of silica gel lubricant dab from the alcohol prep pad container to the configured urethra catheter-entry;
the seventh step is to insert the lubricated catheter tip into the configured urethra catheter-entry;
the eighth step is to push the configured lubricated catheter tip up the configured urethra into the bladder to start urine flow through the configured catheter urine exit;
the ninth step is to continue urine flow until urine flow through the configured catheter urine exit ceases;
the tenth step is to stop pushing the configured lubricated catheter tip when urine flow ceases;
the eleventh step is to insert a configured vent. into the configured catheter urine flow lumen and push the configured vent to being blocked at the catheter tip;
the twelfth step is to withdraw the configured catheter and vent from the configured urethra;
the thirteenth step is to water-rinse the configured catheter and vent and separate and dry the vent and the configured catheter.
